(19) United States
(12) Patent Application Publication
(10) Pub. No.: US 2024/0374623 A1
(43) Pub. Date: Nov. 14, 2024

(54) TKTL1 INHIBITORS FOR ANTIVIRAL THERAPY

(71) Applicant: Tavargenix GmbH, Darmstadt (DE)

(72) Inventors: Johannes F. Coy, Hainburg (DE); Ralf Schierl, Darmstadt-Eberstadt (DE)

(73) Assignee: Tavargenix GmbH, Darmstadt (DE)

(21) Appl. No.: 18/554,636

(22) PCT Filed: Apr. 7, 2022
(86) PCT No.: PCT/DE2022/100262

§ 371 Date: Oct. 10, 2023

(30) Foreign Application Priority Data
Apr. 14, 2021 (DE) 10 2021 109 341.4

(51) Int. Cl. A61K 31/675; A61K 31/05; A61K 31/51; A61P 31/14; C12N 15/113

(52) U.S. Cl. CPC A61K 31/675; A61K 31/05; A61K 31/51; A61P 31/14; C12N 15/1137; C12N 2310/12; C12N 2310/14

(57) ABSTRACT

At least one inhibitor of TKTL1 transketolase is used in patients to treat virus infections, in particular RNA virus infections and more particularly SARS-CoV-2 infections.

The invention relates to inhibitors of the transketolase TKTL1 (hereinafter referred to as

“TKTL1 inhibitor” or “TKTL1 inhibitors” for short) for use in antiviral therapy, in particular therapy of RNA viruses and in particular also of SARS-CoV-2 infections and covid-19 diseases.

In 2019, a new corona virus called SARS-CoV-2 was described in China, infecting people and spreading rapidly. Some of the infected people develop a disease called COVID-19, which damages to the lungs and also causes oxygen deficit and respiratory distress. In March 2020, WHO declared the outbreak of SARS-CoV-2 infections a pandemic. Infection with the SARS-CoV-2 virus, unlike infection with influenza viruses, can also be asymptomatic, which means that apparently healthy people can infect others with SARS-CoV-2 viruses. This massively complicates the containment of SARS-CoV-2 infections. In addition to asymptomatic courses of infection, there are many people with SARS-CoV-2 who have flu-like symptoms such as cough, sore throat, diarrhea, or fever. In addition, unlike influenza, there are also symptoms such as loss of sense of taste and other symptoms or deficits associated with neuronal function. Such neurologically associated symptoms as for example cognitive performance limitations, fatigue, chronic fatigue syndrome, which manifest long after the disappearance of the current SARS-CoV-2 infection, represent a major health problem of infected individuals. A subset of SARS-CoV-2 infected persons also develop pneumonia, sometimes bilateral pneumonia. Multiple organ failure resulting in death may also result from SARS-CoV-2 infection. In such severe courses of SARS-CoV-2 infection, there is often an overreaction, i.e., an excessive response of the immune system, in which, for example, immune cells migrate into the lungs and inhibit their functionality. Drugs that inhibit immune system activity, such as dexamethasone, show positive effects on this disease process. Nevertheless, a high proportion of those patients who have a severe course of SARS-CoV-2 infection die. To date, no sufficiently effective therapies are available to prevent or treat severe courses of covid-19 disease.

In the case of many other viral diseases, too, no sufficiently effective agents are yet available to combat viral infection in patients. For example, there is as yet no adequate therapy for virus-triggered dengue fever or severe forms of herpes infection of the eye that lead to blindness.

Most of the therapeutic control strategies known in the prior art for viral diseases are aimed at rendering the virus itself harmless, e.g. prophylactic therapies using vaccines or palliative and curative therapies using the administration of antiviral agents. These strategies have in common that they attack virus-specific structures (epitopes/enzymes). However, viruses can adapt very quickly to new environmental conditions due to their ability to change their genome within a very short time. This is currently illustrated by the new variants of the SARS-CoV2 virus, which are better able to spread or are better able to protect themselves against the body's own immune system or are better protected against vaccination or even result in an increase in the lethality of infections. Therefore, therapeutic strategies based on virus-specific structures can quickly fail, because among the naturally and numerous virus mutants that have evolved, there are also those that are insensitive to the agents in question or with regard to vaccines. It is also fundamentally true that it is not possible to predict which viral mutation or which virus will emerge next and pose a threat to humans in the future. Timely development and planning of virus-specific therapy strategies is therefore very difficult to almost impossible.

The genome of viruses comprises either RNA or DNA. The SARS-CoV-2 virus belongs to the RNA viruses.

After infection and release of their viral RNA in a cell, RNA viruses depend on the replication (duplication) of this viral RNA to occur rapidly and in as large a quantity as possible. For this replication, ribose-5-phosphate (R5P) from the host cell is absolutely necessary. DNA viruses also require R5P for their replication in the host cell, because deoxyribose-5-phosphate, the essential building block of their DNA, is produced from ribose-5-phosphate. Thus, both RNA viruses and DNA viruses are dependent on R5P synthesis in the host cell.

In mammalian cells, R5P is provided via the pentose-phosphate pathway (PPP). According to current doctrine, this can be carried out via the oxidative part as well as via the non-oxidative part of the PPP.

Transketolases (TKT) are key enzymes of PPP. They form dimers and are activated by the binding of a molecule of thiamine diphosphate and a divalent cation. This transketolase enzyme activity can be altered by forming a heterodimer of a TKT and a transketolase-like-1 (TKTL1) protein (Li et al., 2019).

Li et al. (2019) describe that the formation of TKTL1-TKT heterodimers allows cells to reprogram R5P metabolism: When more R5P is needed, which is especially the case for nucleotide and DNA synthesis during S phase of the cell, increased TKTL1 levels lead to increased formation of TKTL1-TKT heterodimers. These promote R5P production. In other words, and in simplified terms, Li et al. show that the basic R5P supply of a cell is provided by the TKT-TKT homodimer. If the cell requires an additional large amount of R5P, the expression of TKTL1 and the resulting TKT-TKTL1 heterodimer formation will, in a sense, start an “intracellular turbo program” that promotes increased production of R5P to enable cell division and thus cell proliferation.

Studies by Bojkova et al. (2020) using SARS-CoV-2 infected cells of the human cell line Caco-2 have shown that the PPP of the cells is activated in the SARS-CoV-2 infected cells. Bojkova et al. (2020) also investigated whether SARS-CoV-2 replication can be affected by TKT inhibitors. The use of the TKT inhibitor benfooxythiamine (B-OT), a prodrug of the TKT inhibitor oxythiamine (which is an inhibitory analog of thiamine), resulted in inhibition of SARS-CoV-2 virus replication in host cells. However, B-OT at a concentration of at least 5 mM B-OT was required to inhibit SARS-CoV-2 virus replication. Translated to a human, this would correspond to an amount of B-OT to be administered of at least 1 gram of B-OT per kg of body weight, i.e., for a 70 kg human, an amount of 70 grams of B-OT to be administered. This value is outside the normally or even exceptionally used amount of active ingredient, and it is far above the tolerable amounts of oxythiamine known in the prior art for humans, and this may be transferred to B-OT. The skilled person therefore assumes that B-OT and TKT inhibitors per se do not represent a usable option with regard to an active ingredient for virus inhibition.

The present invention is based on the task of providing an agent for combating viral infections, in particular RNA virus infections, which is also particularly suitable for combating SARS-CoV-2 viruses.

One solution to this task is to provide at least one inhibitor of the transketolase TKTL1 (hereinafter referred to as “TKTL1 inhibitor” for short) for use in the medical-therapeutic treatment of viral infections (antiviral therapy), in particular the treatment (therapy) of RNA viral infections and in particular also the treatment (therapy) of SARS-CoV-2 infections and associated diseases such as COVID-19.

In other words, one solution to this task consists in the technical teaching of the use of at least one inhibitor of the transketolase TKTL1 (abbreviated to “TKTL1 inhibitor”) as the active ingredient of an antiviral and, in particular, virostatic (virus-inhibiting) drug.

### DEFINITIONS OF TERMS:

The term “inhibitor” is used herein in context to mean an agent (i.e., in principle, any agent) and, in particular, a substance (or mixture of substances, including pharmaceutical compositions) capable of directly or indirectly effecting/causing a reduction in the activity of the TKTL1 polypeptide in an individual or in cells of an individual. Such agents or material substances include, in particular:
   (a) Inhibitors of transcription of the TKTL1 gene, for example, substances that bind to the TKTL1 promoter and thereby restrict or inhibit its activity, such as resveratrol, which inhibits the TKTL1 promoter in a dose-dependent manner.
   (b) Inhibitors of translation of TKTL1 mRNA, for example, inhibitory RNAs such as antisense constructs, siRNAs, sh-RNAs, ribozymes.
   (c) Inhibitors of the enzyme reaction of TKTL1 and/or of the TKTL1-TKT heterodimer complex, in particular active substances/ingredients that restrict or prevent this enzyme reaction. These include in particular antagonists of cofactors of TKTL1 polypeptides such as antithiamine compounds.
   (d) Inhibitors of the formation of the TKTL1-TKT heterodimer complex, especially agents that restrict or prevent heterodimer formation.
The term “RNA” is synonymous here with ribonucleic acid(s).
The term “DNA” is synonymous here with deoxyribonucleic acid.

The present invention is based on the surprising finding that inhibition of the enzyme TKTL1 and thus inhibition of the enzyme activity of the TKTL1/TKT heterodimer in human cells results in significant inhibition of the replication of the viral RNA or viral DNA, but the host cell itself remains viable and the human organism does not suffer permanent and irreversible damage.

Since the division and thus proliferation of cells is vital for the human organism, it was to be expected that inhibition of TKTL1 and/or the TKTL1/TKT heterodimer in a magnitude that is required according to Bojkova et al. (2020) to slow down or stop viral proliferation would lead to inhibition of cell division and thus to severe side effects including death. Also, and precisely because transketolases and especially TKTL1 are considered fundamentally relevant for cell viability as a key enzyme in the regulation of cell cycle and cell division, they appeared unsuitable as potential targets for an antiviral therapy strategy according to common doctrine because severe side effects were to be expected.

Surprisingly, however, it was found that inhibition of the enzyme activity of the TKTL1/TKT heterodimer in virus-infected human host cells results in the prevention or significant inhibition of the massive (strong) increase in ribose-5-phosphate (R5P) production via the host cell metabolism required for virus nucleic acid biosynthesis and thus for the propagation of virus nucleic acid (RNA or DNA), but on the other hand the amounts of R5P required for the survival of the host cell are maintained.

In other words, the use of TKTL1 inhibitors in the treatment of viral infections and in particular also of SARS-CoV-2 infections is based on the surprising finding that the biosynthesis of viral nucleic acid and thus viral replication in the human host cell can be significantly inhibited or reduced by TKTL1 inhibitors without endangering the survival of the host cell and thus without any risk of damage to the human body.

The use of the TKTL1 inhibitor according to the invention interferes with the metabolism of the host cell and thus of the entire human body and has the effect that there are no longer sufficient ribose-5-phosphate building blocks (R5P) available for virus replication and thus virus replication is slowed down or even completely prevented without irreversibly damaging the cell and the entire human body. This is accompanied by the advantage that the virus itself is not (directly) the active substance target, but rather the host cell metabolism, and that therefore any virus mutations do not impair the success of the application according to the invention.

The use of TKTL1 inhibitors according to the invention represents a therapeutic strategy that does not address virus-specific structures, but rather factors that originate from the infected host cell and are required by the virus for its replication. This provides an antiviral therapy strategy which, firstly, can be used promisingly against a broad spectrum of viruses and, secondly, is also effective against mutations of the virus (virus mutants). This also allows protection against viruses that will only emerge in the future. The invention described allows protection against viruses without knowing their RNA or DNA sequence. Thus, in comparison to a vaccination, no lead time is required for the development of a drug, so that a therapy exists immediately. This enables the protection of humanity and mammals in general from existing and future viruses and diseases that originate and will originate from them. For the first time, there is existence protection against viral diseases.

In a preferred embodiment, the inhibitor is a substance or mixture of substances that is capable and suitable of specifically restricting or preventing the transcription of the TKTL1 gene. In particular, a substance capable of and suitable for binding to the TKTL1 promoter of the cell and thereby restricting or preventing its activity is suitable for this purpose. In practice, resveratrol in particular has proven to be a suitable inhibitor of the TKTL1 promoter (Kumar B, 2018).

In a likewise preferred embodiment, the inhibitor is a substance or mixture of substances suitable for specifically inhibiting the translation of TKTL1 mRNA. In particular, at least one substance selected from the group of TKTL1 mRNA-specific antisense constructs, TKTL1 mRNA-specific siRNAs, TKTL1 mRNA-specific sh-RNAs, TKTL1 mRNA-specific ribozymes and other TKTL1 mRNA-specific inhibitory RNAs is suitable for this purpose.

In a further preferred embodiment, the inhibitor is a substance or mixture of substances suitable for restricting or inhibiting the enzyme reaction of TKTL1 and/or of the TKTL1-TKT heterodimer complex. In particular, at least one substance selected from the group of antithiamine compounds and other antagonists, in particular antagonists from the group of cofactors of the enzyme TKTL1, is suitable for this purpose.

In one embodiment tested in practice, the TKTL1 inhibitor is an inhibitory thiamine analog. All previously known transketolase enzymes, including TKTL1, are functionally dependent on thiamine (vitamin B1) as a coenzyme. Thiamine analogs such as oxythiamine act as thiamine antagonists and can be used to inhibit transketolases (see EP1354961A1).

A preferred inhibitory thiamine analog is the substance benfo-oxythiamine (B-OT). B-OT is a precursor (“pro-pharmacon”, “prodrug”) of oxythiamine, can be applied orally and releases oxythiamine shortly after its uptake into the mammalian organism. Via the bloodstream, B-OT can reach all cells in all parts of the body. In vivo pharmacokinetics data have shown that oxythiamine can penetrate the blood-brain barrier and consequently can be used with promise in viral infections of the brain.

The chemical structure (structural formula) of benfo-oxythiamine is known, e.g. from EP1354961A1.

Surprisingly, it was found that a concentration of B-OT more than 1000-fold lower than the minimum concentration described by Bojkova et al. (2020) for human Caco-2 cell lines at the cell culture level is sufficient for inhibition of the enzyme activity of TKTL1 in the TKTL1/TKT heterodimer in the human organism. In their studies, Bojkova et al. (2020) show that at least a 5 mM concentration of B-OT is required to inhibit SARS-CoV-2 virus replication in SARS-CoV-2 infected Caco-2 cell lines. Unexpectedly and surprisingly to those skilled in the art, however, during the course of the studies upon which the present invention is based, it has been shown that administration of less than 35 mg of B-OT per day per 70 kg of patient body weight, i.e., less than 0.5 mg per kg of body weight per day and, accordingly, less than a 1 nanomolar (1 nM) concentration of B-OT per kg of body weight per day, is sufficient to inhibit SARS-CoV-2 virus replication in the human body.

Therefore, according to the invention, in the case of benfo-oxythiamine as TKTL1 inhibitor, the BOT is applied/administered in a dose/dosage of 7 μg to 430 μg (μg=micrograms), preferably 14 μg to 215 μg, more preferably 14 μg to 130 μg B-OT per kg body weight of the patient and per day. In other words, the dose/dosage of the TKTL1 inhibitor benfo-oxythiamine per kg body weight of the patient and per day is 7 μg≤μg B-OT≤430 μg, preferably 14 μg≤μg B-OT≤215 μg, and particularly preferably 14 μg≤μg B-OT≤130 μg.

In general, according to the invention, in the case of benfo-oxythiamine as a TKTL1 inhibitor, the benfo-oxythiamine is used in a dose of less than 0.5 mg, preferably less than 0.3 mg per kg body weight of the patient and per day.

The production of Benfo-Oxythiamine (B-OT) according to the EU GMP Guide for Human and Veterinary Medicinal Products is well established in the state of the art, which allows the use of Benfo-Oxythiamine in mammals (e.g. dogs, cats) and especially also in humans.

In a further preferred embodiment, the TKTL1 inhibitor is a substance or mixture of substances suitable for limiting or preventing the formation of the TKTL1-TKT heterodimer complex.

The invention is explained in more detail below by means of embodiment with tables and figures.

In the figures show:

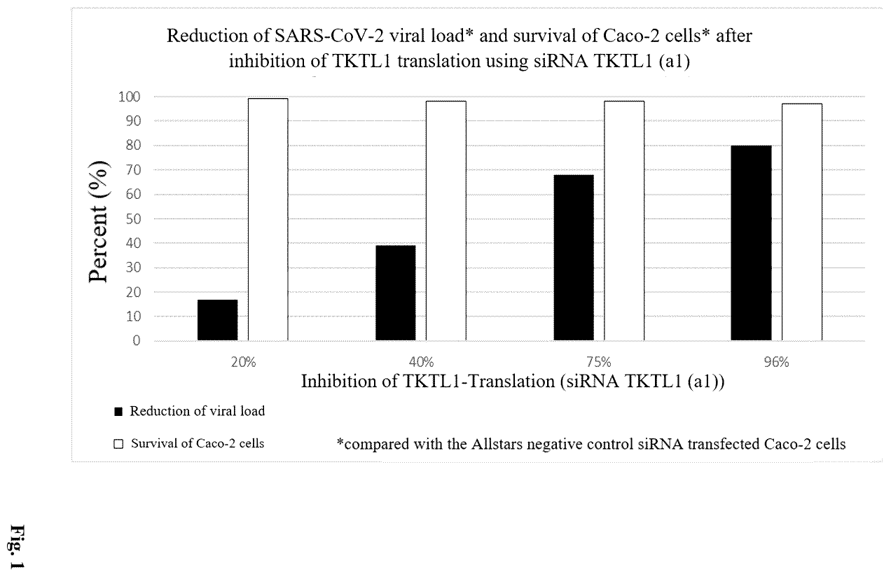

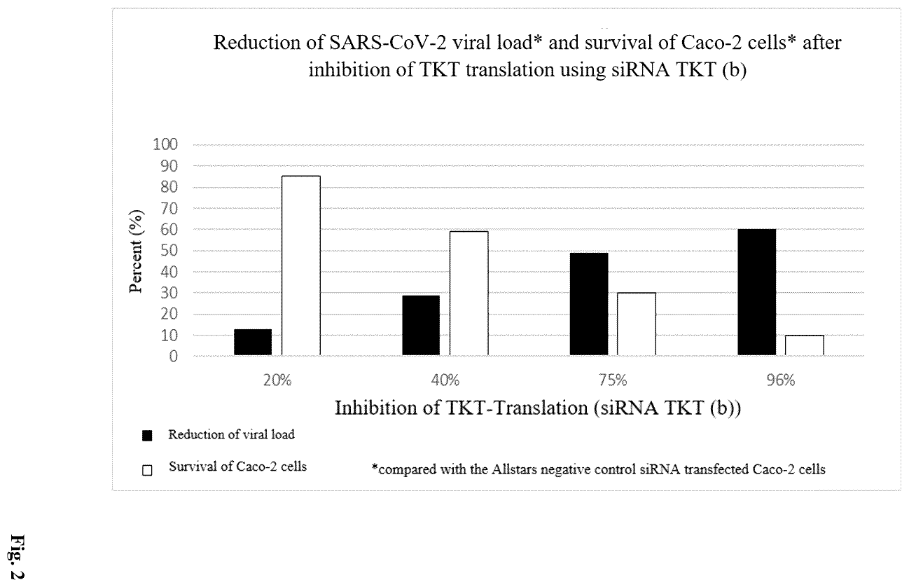

FIG. 1: The reduction of SARS CoV-2 viral load and survival of Caco-2 cells after inhibition of TKTL1 translation using TKTL1-specific siRNA.

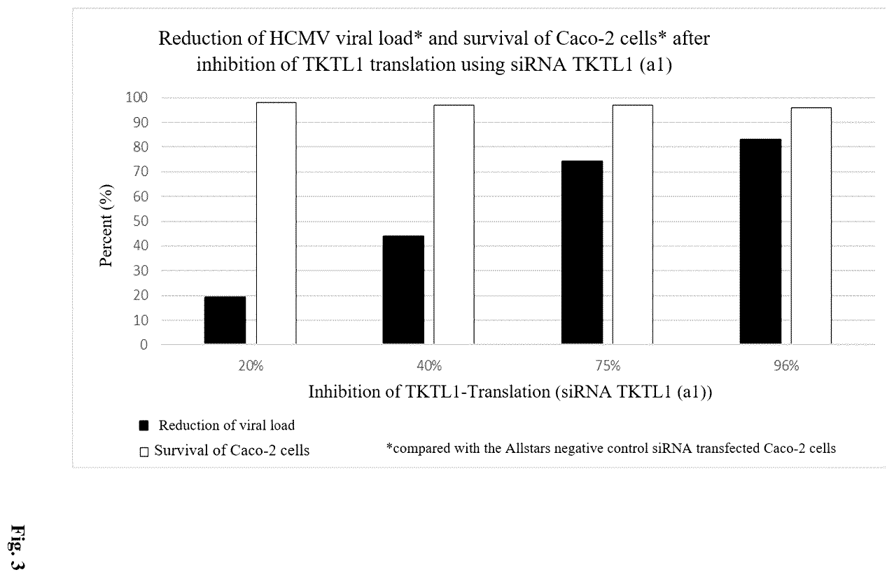

FIG. 2: The reduction of SARS CoV-2 viral load and survival of Caco-2 cells after inhibition of TKT translation using TKT-specific siRNA.

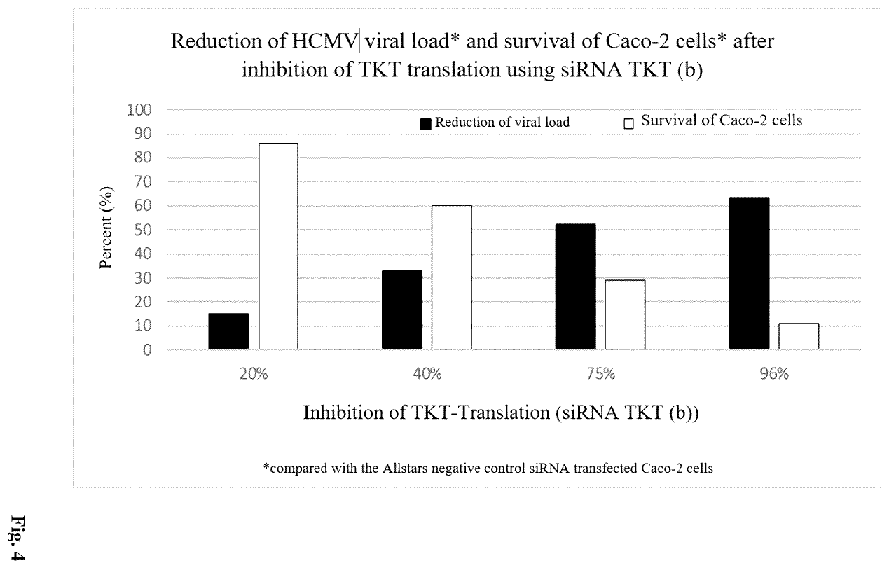

FIG. 3: The reduction of human cytomegalovirus (HCMV) viral load and survival of Caco-2 cells after inhibition of TKTL1 translation using TKTL1-specific siRNA.

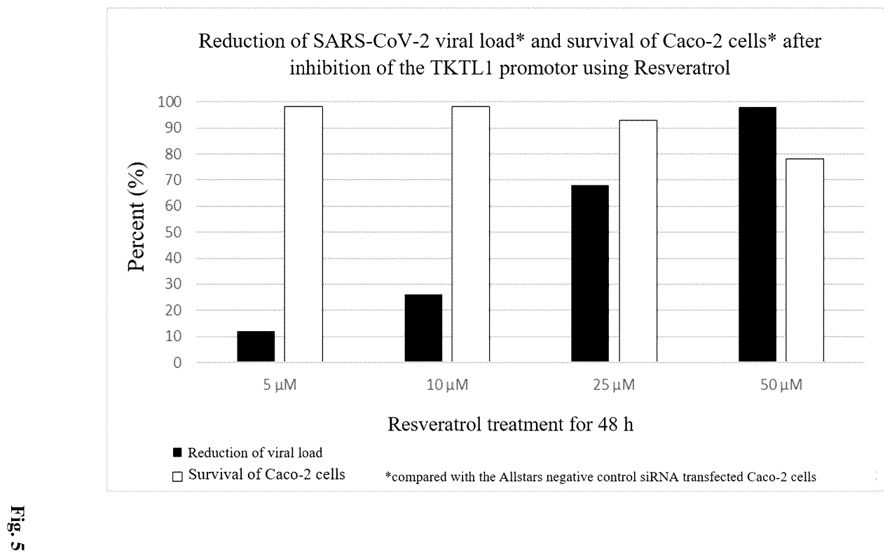

FIG. 4: The reduction of human cytomegalovirus (HCMV) viral load and survival of Caco-2 cells after inhibition of TKT translation using TKT-specific siRNA.

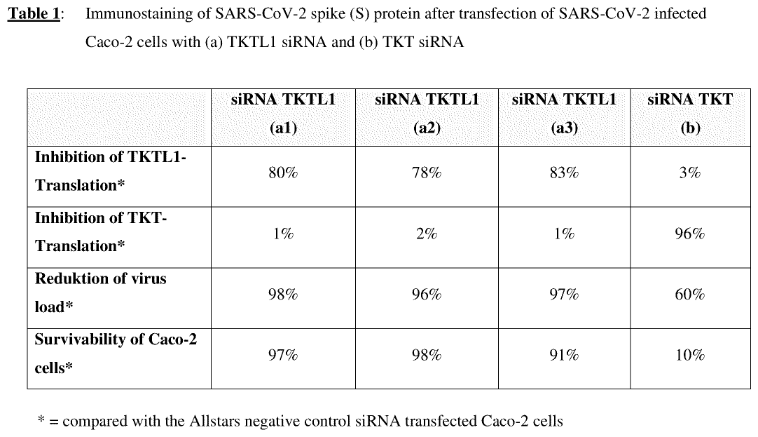

FIG. 5: The dose-dependent reduction of SARS Cov-2 viral load and survival of Caco-2 cells after inhibition of TKTL 1 promoter using different doses of resveratrol.

The reduction in viral load is shown in each case as a reduction by the percentage value indicated.

### Example 1: siRNA Inhibition of (a) TKTL1 Compared to siRNA Inhibition of (b) TKT in SARS-CoV-2 Infected Mammalian Cells

Using a transfection reagent comprising polycationic and neutral lipids (here METAFECTENE® from Biontex Laboratories GmbH, Munich, Germany) SARS-CoV-2 infected Caco-2 cells were transfected with (a) human TKTL1-specific siRNAs and (b) human TKT-specific siRNAs and with (c) an siRNA negative control (here the AllStars negative control siRNA from Qiagen, which according to the manufacturer's instructions has no homology to any known mammalian gene). (siRNA or small interfering RNA are short RNA molecules that do not code for proteins but bind to complementary single-stranded RNA molecules, thereby preventing the latter from functioning normally).

The following siRNA sequences were used:

(a) TKTL1 specific:
1.
5′-GGAGUUGCAUGUGGAAUGG-3′
(Diaz-Morelli et al. 2016).
2.
5′-UUAUUCACGAAGGAAACACUU-3′
(Heller et al. 2018).
3.
5′-UAAAUAACCAUAGUUUCUGGU-3′
(Heller et al. 2018).
(b) TKT specific:
5′-AAUGAUGGCUGUUGGCUGGTT-3′
(Lu et al. 2017).

Effective silencing (i.e., gene silencing, gene expression knockdown by RNA interference) for (a) TKTL1 and (b) TKT in the SARS-CoV-2 infected and siRNA-transfected Caco-2 cells was demonstrated by RTqPCR (reverse transcriptase quantitative PCR) and Western blot analyses.

To determine and quantify TKTL1/TKT mRNAs and TKTL1/TKT proteins, RTqPCR (reverse transcriptase quantitative PCR) and Western blot analyses were performed 72 h and 96 h after transfection in the SARS-CoV-2 infected and siRNA-transfected Caco-2 cells.

The replication ability of SARS-CoV-2 viruses after transfection of Caco-2 cells with the different siRNAs was determined by immunostaining for SARS-CoV-2 spike(S) protein. Spike protein staining was used to calculate the percentage of virus inhibition in cells transfected with (a) TKTL1 or (b) TKT siRNA compared with (c) cells transfected with the siRNA negative control.

Cell viability of SARS-CoV-2-infected Caco-2 cells transfected with the different siRNA were assessed by methylthiazolyldiphenyl-tetrazolium bromide (MTT) assay.

The results of these tests are shown in Table 1 and FIGS. 1 and 2.

Depending on the different siRNA sequences, the following effects were observed in terms of reduction of viral load and survivability of SARS-CoV-2 infected Caco-2 cells compared to Caco-2 cells transfected with AllStars negative control siRNA: In cells transfected with TKTL1-specific siRNA (compared with cells transfected with nontargeted control duplexes), inhibition of TKTL1 translation occurred. Thereby, inhibition of TKTL1 translation at a level at which cells remained viable resulted in a significant reduction in the replicative capacity of SARS-CoV-2 viruses.

Consequently, these experiments show that inhibition of TKTL1 to a degree that does not affect cell survival can significantly inhibit SARS-CoV-2 virus replication.

The parallel approaches using TKT-specific siRNA as an inhibitor of translation of TKT mRNA show that inhibition of TKT translation also leads to a significant reduction in viral load in SARS-CoV-2 infected Caco-2 cells. However, it also reduces cell viability to a significant extent. This indicates that the human therapeutic window of inhibition of TKT for inhibition of

SARS-CoV-2 virus replication greatly overlaps with human cell damage and thus practically does not exist. In other words, the extent of inhibition of TKT necessary to cause inhibition of SARS-CoV-2 virus replication is so great that the human cell is damaged to the point of death. Administration of an inhibitor of transketolase TKT at doses that result in inhibition of SARS-CoV-2 virus replication would cause such severe damage in the human body that therapeutic use of TKT inhibition for the purpose of inhibiting SARS-CoV-2 virus replication in the human body must be ruled out.

Conclusion: The results show that the application of TKTL1-specific siRNA as an inhibitor of TKTL1 in SARS-CoV-2 infected Caco-2 cells has the effect of preventing virus multiplication in host cells, and the host cells survive in the process.

Parallel studies in vitro with human cytomegalovirus (HMCV) (see Example 2 here) confirm that the measure of siRNA inhibition of TKTL1 can prevent virus multiplication in host cells without compromising host cell survival.

### Example 2: siRNA Inhibition of (a) TKTL1 Compared to siRNA Inhibition of (b) TKT in Mammalian Cells Infected With Human Cytomegalovirus (HCMV)

The effects of siRNA inhibition of TKTL1 on the one hand and TKT on the other hand were investigated in Caco-2 cells infected with human cytomegalovirus (HCMV). Recombinant HCMV EYFP viruses, which also encode enhanced yellow fluorescent protein (EYFP), were used to infect Caco-2 cells as described by Dal Pozzo et al. (2008). Thus, the replication capacity of HCM virus can be determined using fluorescence-based antiviral assays (e.g., Dal Pozzo et al., 2008).

Caco-2 cells were transfected with the siRNA sequences for TKTL1 and TKT given in Example 1. Effective silencing was demonstrated by RTqPCR (reverse transcriptase quantitative PCR) and Western blot analyses. These analyses were performed 72 h and 96 h after transfection. Viability of HMCV-infected cells was determined by MMT assay.

The replication capacity of HCM viruses after transfection of Caco-2 cells with the different siRNAs was determined by fluorescence-based antiviral assay (Dal Pozzo et al 2008). Based on the EYFP signals of the recombinant viruses, the percentage of virus inhibition was calculated in the cells transfected with (a) TKTL1 or (b) TKT siRNA compared with (c) cells transfected with the siRNA negative control. The results are shown in FIGS. 3 and 4.

These results confirm those obtained in Example 1 with SARS-CoV-2 also for HMCV-infected cells: Inhibition of TKTL1 using TKTL1-specific siRNA as an inhibitor causes the HMC viruses to stop replicating in the host cells and the host cells survive in the process. In contrast, TKT inhibition using TKT-specific siRNA also leads to a significant reduction in viral load in HMCV-infected Caco-2 cells, but with significantly reduced cell viability.

### Example 3: Use of B-OT as a TKTL1 Inhibitor in Mammalian Cells Infected With Human Cytomegalovirus (HCMV)

Furthermore, the sensitivity of HCMV replication to B-OT in human mammalian cells was investigated. For this purpose, experiments were performed as described in the work of Dal Pozzo et al. 2008 with B-OT: Human embryonic lung fibroblasts (HEL) were infected with recombinant HCMV EYFP viruses additionally encoding enhanced yellow fluorescent protein (EYFP) (Dal Pozzo F, et al., 2008). As a measure of viral replication after infection with HCMV-EYFP, the emission of the EYFP protein was measured (excitation wavelength: 485 nm/emission: 530 nm). The constant and low cell-associated background emission measured in an automated manner was subtracted from the fluorescence emission value. To test the antiviral effect of B-OT, HCMV-EYFP-infected cells were incubated for 48 h with different B-OT concentrations (0 to 20 mM B-OT). Dose-dependent inhibition of fluorescence (as a measure of viral replication) was evident. The required compound concentration that reduced EYFP emission by half compared with untreated controls (0 mM B-OT) was defined as the 50% inhibitory concentration (IC50) in the fluorescence-based antiviral assay. The IC50 determined here was 0.5 mM B-OT.

### Example 4: Use of B-OT as a TKTL1 Inhibitor to Inhibit SARS-CoV-2 Virus Replication in the Human Body

It is known from Zhu et al 2020 that in patients with SARS-CoV-2 infection and Covid-19 disease, assessment of disease severity can be based on clinical levels of immune-inflammatory markers. High levels of the proinflammatory cytokine IL-6 and/or C-reactive protein (CRP) indicate severe disease and a high-risk disease course. The proinflammatory cytokine IL-6 plays a key role in the “cytokine storm” described for patients with SARS-CoV-2 infections due to its pleiotropic nature. Its constitutive expression causes organ damage and severe pain (Zhu et al 2020; Gupta et al 2020).

In the context of individual curative trials on hospitalized patients with SARS-CoV-2 infection and COVID-19 disease, it was unexpectedly and surprisingly found by those skilled in the art that a daily administration of less than 20 mg of B-OT per 70 kg of patient body weight, i.e. less than 0.3 mg per kg of body weight and, accordingly, less than a 1 nanomolar (1 nM) concentration of B-OT per kg of body weight, over the course of 7 days resulted not only in a significant reduction in viral load, as detected by PCR, but also in a significant reduction in the inflammatory parameters C-reactive protein (CRP) and interleukin-6 (IL-6): under 7-day B-OT therapy, CRP levels dropped on average from 77 pg/ml to 5 pg/ml, while IL-6 levels dropped from an average of 63 pg/ml to 5 pg/ml.

### Example 5: Use of Resveratrol as a TKTL1 Inhibitor to Inhibit Viral Replication in SARS-CoV-2 Infected Caco-2 cells

The organic compound resveratrol (molecular formula C₁₄H₁₂O₃) from the group of polyphenols is known to the skilled person as an inhibitor of the TKTL1 promoter. In Kumar B. (2018), it is described that treatment of HeLa cells with 50 M resveratrol for 48 h resulted in a 75% reduction in TKTL1 promoter activity compared to untreated cells. However, at the same time, this also led to a significant decrease in the viability of HeLa cells.

In the studies in this Example 5, resveratrol was used to inhibit virus replication in SARS-CoV-2 infected Caco-2 cells. For this purpose, the SARS-CoV-2 infected Caco-2 cells were treated with different resveratrol concentrations for 24 h. The resveratrol concentration was determined by the following experiments. To confirm the effect of reduced promoter activity on TKTL1 mRNA production, RTqPCR was performed. The results of this RTqPCR show a reduction of TKTL1 mRNA depending on the resveratrol concentration applied: the higher the concentration the stronger the reduction. Furthermore, as described here in Example 1, the survivability of Caco-2 cells was determined by MTT assay and the replication ability of SARS-CoV-2 viruses was determined by immunostaining for the SARS-CoV-2 spike (S) protein. SARS-CoV-2-infected Caco-2 cells without resveratrol treatment served as controls.

It was found that the replication ability of SARS-CoV-2 viruses under resveratrol treatment decreased with increasing resveratrol dose compared to control, while the viability of SARS- CoV-2-infected Caco-2 cells remained at a sufficient level. Thus, under treatment with 25 μM resveratrol for 48 h, the replication ability of SARS-CoV-2 viruses decreased by 68% compared to the control, while the viability of Caco-2 cells was 93%. The results are shown graphically in FIG. 5.

### CITED NON-PATENT LITERATURE:

Bojkova D, Costa R, Bechtel M, Ciesek S, Michaelis M, Cinatl Jr J (2020):
Targeting pentose phosphate pathway for SARS-CoV-2 therapy.
bioRxiv preprint https://doi.org/10.1101/2020.08.19.257022
Dal Pozzo F, Andrei G, Daelemans D, Winkler M, Piette J, De Clercq E, Snoeck R, (2008):
Fluorescence-based antiviral assay for the evaluation of compounds against vaccinia virus, varicella zoster virus and human cytomegalovirus. Journal of Virological Methods 151 66-73.
doi: 10.1016/j.jviromet.2008.03.025
Diaz-Moralli S, Aguilar E, Marin S, Coy J F, Dewerchin M, Antoniewicz M R, Meca-Cortés O, Notebaert L, Ghesquière B, Eelen G, Thomson T M, Carmeliet P, Cascante M. (2016):
A key role for transketolase-like 1 in tumor metabolic reprogramming.
Oncotarget 7(32): 51875-51897; doi: 10.18632/oncotarget. 10429; PMID: 27391434.
Gupta K. K, Khan M. A, Singh S. K (2020): Constitutive Inflammatory Cytokine Storm:
A Major Threat to Human Health.
Journal of Interferon & Cytokine Research. January 2020.19-23.
http://doi.org/10.1089/jir.2019.0085
Heller S, Maurer G D, Wanka C, Hofmann U, Luger A L, Bruns I, Steinbach J P, Rieger J.
(2018): Gene Suppression of Transketolase-Like Protein 1 (TKTL1) Sensitizes Glioma Cells to hypoxia and Ionizing Radiation.
Int J Mol Sci. 2018 Jul. 25;19 (8):2168.
Kumar B (2018): Resveratrol inhibits expression of cancer-specific pentose phosphate pathway enzyme TKTL1. Asian Journal of Pharmaceutical and Clinical Research 11:332.
DOI: 10.22159/ajpcr.2018.v11i6.25021
Li Y, Yao C F, Xu F J, Qu Y Y., Li J T, Lin Y, Cao Z L, Lin P C, Xu W, Zhao S M, & Zhao J Y
(2019): APC/C^{CDH1} synchronizes ribose-5-phosphate levels and DNA synthesis to cell cycle progression.
Nature Communications (2019) 10:2502; https://doi.org/10.1038/s41467-019-10375-x
Ln H, Zhu H. (2017): Effect of siRNA-mediated gene silencing of transketolase on A549 lung cancer cells. Oncol Lett. 2017; 14 (5): 5906-5912. DOI: 10.3892/ol.2017.6916.
Zhu Z, Cai T, Fan L, Lou K, Hua X, Huang Z, Gao G. (2010): Clinical value of immune-inflammatory parameters to assess the severity of coronavirus disease 2019.
Int J Infect Dis. 2020 June; 95: 332-339. http://doi: 10.1016/j.ijid.2020.04.041

1. A method for treating a viral infection comprising: administering to a patient in need thereof an inhibitor of transketolase TKTL1 (transkeolase-like-1) in an amount effective for treating the viral infection.

2. The method according to claim 1, wherein a substance or mixture of substances is/are administered which is/are adapted to restrict or prevent transcription of TKTL1 gene.

3. The method according to claim 1, wherein a substance or mixture of substances is/are administered which are adapted to bind to a TKTL1 promoter of a cell of the patient and thereby restricting or inhibiting an activity of the TKTL1 promoter.

4. The method according to claim 3, wherein the substance is resveratrol.

5. The method according to claim 1, wherein a substance or mixture of substances is/are administered which is/are adapted to inhibit translation of TKTL1 mRNA.

6. The method of claim 5, wherein the substance or mixture of substances is/are inhibitory RNA(s) specific for TKTL1 mRNA.

7. The method according to claim 1, a substance or mixture of substances is/are administered which is/are adapted to restrict or prevent an enzyme reaction of the TKTL1 and/or a TKTL1-TKT heterodimer complex.

8. The method of claim 7, wherein the substance or mixture of substances is/are antagonists of cofactors of TKTL1 polypeptides.

9. The method according to claim 7 wherein the substance is an inhibitory thiamine analog.

10. The method according to claim 9, wherein the inhibitory thiamine analog is benfo-oxythiamine and is used in a dose of 7 μg to 430 μg per kg body weight of the patient and per day.

11. The method according to claim 1, wherein a substance or mixture of substances is/are administered which is/are adapted to restrict or prevent formation of a TKTL1-TKT heterodimer complex.

12. The method of claims 1, wherein the inhibitor is benfo-oxythiamine as and is administered in a dose of 14 μg to 215 μg per kg body weight of the patient and per day.

13. The method according according to claim 12, wherein the dose per kg body weight of the patient and per day is 14 μg to 130 μg benfo-oxythiamine.

14. The method of claim 1, wherein the viral infection is a RNA virus infection.

15. The method of claim 14, the RNA virus is the SARS-CoV-2 virus.

16. The method of claim 6, wherein the inhibitory RNAs are antisense RNAs, siRNAs and/or ribozymes.

17. The method of claim 8, wherein the antagonists are antithiamine compounds.

18. The method of claim 9, wherein the inhibitory thiamine analog is oxythiamine, benfo-oxythiamine and/or a benfo-oxythiamine analog.
